(19) United States
(12) Patent Application Publication
(10) Pub. No.: US 2021/0060354 A1
(43) Pub. Date: Mar. 4, 2021

(54) METHODS FOR PREVENTING AND TREATING DIABETIC RETINOPATHY AND DIABETIC MACULAR EDEMA

(71) Applicant: University of Wisconsin-Milwaukee Research Foundation, Inc., Milwaukee, WI (US)

(72) Inventors: Janis T. Eells, Milwaukee, WI (US); Elizabeth S. Liedhegner, Milwaukee, WI (US)

(21) Appl. No.: 17/013,444

(22) Filed: Sep. 4, 2020

Related U.S. Application Data
(60) Provisional application No. 62/895,908, filed on Sep. 4, 2019.

(51) Int. Cl. A61N 5/06

(52) U.S. Cl. CPC A61N 5/0622; A61B 5/0496

(57) ABSTRACT

The present disclosure provides methods for preventing or treating ocular diseases such as diabetic retinopathy and diabetic macular edema, wherein the methods include administering non-invasive photobiomodulation light therapy comprising a light having a wavelength in a red wavelength range. Biomarkers and diagnostic assays for selecting a treatment are also provided.

### BACKGROUND

Diabetic retinopathy (DR) is the most common complication of diabetes mellitus and is a leading cause of blindness. The pathophysiology of DR involves inflammation, oxidative stress and vascular degeneration, which can cause bleeding and leakage, distorting vision. Diabetic macular edema (DME) is a common consequence of DR. Like DR, the pathophysiology of DME involves oxidative stress, elevated concentrations of vascular endothelial growth factor (VEGF) and a breakdown of the inner blood-retinal barrier, resulting in extracellular fluid accumulation in macula, resulting in decreased vision. Current interventions for DR and DME include intraocular injection of anti-angiogenic agents (anti-VEGF, such as bevacizumab and ranibizumab) and corticosteroids. However, these interventions are invasive, expensive, and often ineffective.

Accordingly, new modalities for preventing and/or treating DR and DME are needed. The presently disclosed embodiments address these needs and provide other related advantages.

### BRIEF DESCRIPTION OF THE DRAWINGS

FIG. 1 shows that photobiomodulation (PBM) comprising red light diminishes oxidative stress under high glucose conditions in Müller glial cells. Bar graph displaying ROS production in Müller glial cells. Cells were cultured in either normal glucose or high glucose medium for 24 hours. 670 nm or sham control (using a non-illuminated LED array) was applied to the cells at 4.5 J/cm². Oxidative stress was analyzed using DCF-DA. ***p<0.001, ****p<0.0001. n=3, done in triplicate.

FIGS. 2A-2C show that photobiomodulation (PBM) comprising red light restores mitochondrial function under high glucose conditions. Briefly, Müller glial cells were cultured in either normal glucose or high glucose medium for 24 hours. 670 nm or sham control was applied to the cells at 4.5 J/cm². All assays were normalized to the normal glucose sham levels. FIG. 2A shows analysis of NADPH-dependent oxidoreductase activity using MTT. FIG. 2B shows mitochondrial membrane potential analyzed using TMRE. FIG. 2C shows ATP levels analyzed via CellTiter-Glo (Promega®) assay. For each of FIGS. 2A-2C: n=3, (p=0.10). *p<0.05, **p<0.01, ****p<0.0001.

FIG. 3 shows that photobiomodulation (PBM) comprising red light diminishes NFκB activity under high glucose conditions in Müller glial cells. Müller glial cells were cultured in either normal glucose or high glucose medium for 72 hours. 670 nm or sham control was applied to the cells at 4.5 J/cm² daily. NFκB activity was analyzed via reporter gene assay, normalized to the transfection control, Renilla. ***p<0.001 n=4, performed in duplicate.

FIGS. 4A and 4B show that photobiomodulation (PBM) comprising red light suppresses downstream mediators of inflammatory signaling. Müller glial cells were cultured in either normal glucose or high glucose medium for 72 hours. 670 nm or sham control was applied to the cells at 4.5 J/cm² daily while in culture. (FIG. 4A) VEGF mRNA was analyzed via qPCR and normalized to actin. mRNA levels were calculated by ΔΔCt and normalized to Normal glucose sham, n=3, done in duplicate. p=0.0147 (FIG. 4B) ICAM was analyzed via western blot and normalized to GAPDH as the loading control via ImageJ. Inset includes a representative western blot image, n=4. *p<0.05, **p<0.01, ***p<0.001.

### DETAILED DESCRIPTION

In certain aspects, the present disclosure provides methods for preventing and/or treating DR and/or DME, which methods include use of photobiomodulation therapy (PBM) comprising light in a red wavelength range, for example having a wavelength of 670 nm±50 nm, as provided herein.

In the present disclosure, it was surprisingly found that PBM therapy comprising light in a red wavelength range can rescue or prevent inflammatory phenotypes associated with DR in retinal Müller glial cells, which are known to play a primary role in DR onset and progression. Briefly, Müller glial cells were cultured in hyperglycemic conditions to simulate a diabetic state, and treatment with a clinically relevant dose of PBM therapy (670 nm) significantly decreased levels of several inflammatory markers, including production of reactive oxygen species (ROS), NFκB transcriptional activity, and intercellular adhesion molecule-1 (ICAM-1). Further, mitochondrial membrane potential and NADPH-dependent oxidoreductase activity were significantly increased.

Accordingly, PBM therapy comprising light having a wavelength in a red wavelength range can prevent or delay onset and/or progression of DR and/or DME. Subjects at-risk for developing or progressing DR and/or DME can be treated using methods according to the present disclosure.

In some aspects, the present disclosure provides methods for preventing (e.g., permanently) or delaying (e.g., for a statistically significant amount of time, as compared to a onset and/or progression of the disease in a reference subject) onset and/or progression of (i) diabetic retinopathy and/or (ii) diabetic macular edema in an eye of a subject, the method comprising administering to the eye an effective amount of photobiomodulation (PBM) light comprising light having a wavelength in a red wavelength range.

It will be understood that a reference subject can be: (i) a subject of a same or a similar: age or age group; gender; ethnic group; overweight or obese status; diabetic, pre-diabetic, or hyperglycemic status; diagnosis or lack of diagnosis of DR and/or DMR; family history regarding ocular disease and/or hyperglycemia and/or diabetes; and/or general health as the subject administered PBM according to a currently disclosed method; and/or (ii) a typical or average subject within a population (e.g., local, regional, or national), including within a population defined according to: age or age group; gender; ethnic group; overweight or obese status; diabetic, pre-diabetic, or hyperglycemic status; family history regarding ocular disease and/or hyperglycemia and/or diabetes; or diagnosis or lack of diagnosis of DR and/or DMR). A reference subject does not receive PBM according to the present disclosure, and, in certain embodiments, does not receive prophylaxis or treatment for DR and/or DME.

In some aspects, the present disclosure provides methods for treating (i) diabetic retinopathy (DR) and/or (ii) diabetic macular edema (DME) in an eye of a subject, the methods comprising administering to the eye an effective amount of photobiomodulation (PBM) light comprising light having a wavelength in a red wavelength range.

In some aspects, the present disclosure provides methods for (i) reducing or modulating, in an eye of a subject: (a) oxidative stress; (b) NFκB activity; and/or (c) ICAM-1 expression or activity; and/or (ii) increasing or modulating, in an eye of a subject, a mitochondrial membrane potential, a NADPH-dependent oxidoreductase activity level, or both, the methods comprising administering to the eye an effective amount of photobiomodulation (PBM) light comprising light having a wavelength in a red wavelength range.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as size or thickness, or length of time (e.g., seconds, minutes, hours, days, weeks, months) is to be understood to include any integer within the recited range, unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

The term “consisting essentially of” is not equivalent to “comprising”, and refers to the specified materials or steps, or to those that do not materially affect the basic characteristics of a claimed invention.

The terms “treat” and “treatment” refer to medical management of a disease, disorder, or condition of a subject (i.e., patient, host, who may be a human or non-human animal) (see, e.g., Stedman's Medical Dictionary). In general, an appropriate dose and treatment regimen according to the methods and compositions described herein results in a therapeutic or prophylactic benefit. Therapeutic or prophylactic benefit resulting from therapeutic treatment or prophylactic or preventative methods include, for example, an improved clinical outcome, wherein the object is to prevent, delay, retard, or otherwise reduce or limit (e.g., decrease in a statistically significant manner relative to an untreated control) an undesired physiological change or disorder, or to prevent, delay, retard, or otherwise reduce or limit the expansion or severity of such a disease or disorder. Beneficial or desired clinical results from treating a subject include abatement, lessening, or alleviation of symptoms that result from or are associated with the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease onset and/or progression; amelioration or palliation of the disease state; and remission (whether partial or total), whether detectable or undetectable; or overall survival.

“Effective amount” or “therapeutically effective amount” refers to an amount of a composition, combination, or PBM which, when administered to a mammal (e.g., human), is sufficient to aid in preventing or treating a disease (or onset or progression thereof). The amount of the composition or PBM that constitutes an effective amount will vary depending on the condition to be treated and its severity, the manner of administration, and the age of the mammal to be treated, but can be determined routinely by one of ordinary skill in the art having regard to her own knowledge and to this disclosure. Where the intended treatment is prophylaxis, the terms “therapeutically effective” and “prophylactically effective” may be used interchangeably, and may also be used interchangeably with “effective”. When referring to an individual active component (e.g., light of a PBM wavelength or wavelengths), administered alone, an effective dose refers to that component alone. When referring to a combination, an effective dose refers to combined amounts of the active components (e.g., light of different wavelengths) that result in the therapeutic effect, whether administered serially, concurrently or simultaneously. Exemplary effective amounts of PBM for prophylaxis or treatment of DR and/or DME are provided herein.

As used herein, “administration” of a composition or therapy refers to delivering the same to a subject, regardless of the route or mode of delivery. Administration may be effected a single time, continuously (i.e., without stopping, or at regular intervals without a predetermined end), or intermittently. Administration may be for treating a subject already confirmed as having a recognized condition, disease or disease state, or for treating a subject susceptible to or at risk of developing such a condition, disease or disease state. Methods, devices, and parameters for administering PBM include those provided herein.

Subjects in need of the methods described herein include those who already have the disease or disorder, as well as subjects prone to have, or at risk of developing, the disease or disorder. Subjects in need of prophylactic treatment include subjects in whom the disease, condition, or disorder is to be prevented (i.e., decreasing the likelihood of occurrence, or recurrence, of the disease or disorder, or altogether preventing occurrence or recurrence). Clinical benefit provided by the methods described herein can be evaluated using in vitro assays, preclinical studies, and clinical studies in subjects to whom administration of the methods is intended to benefit, as described herein.

Treatment of an ocular disorder (e.g., DR or DME) in an eye or eyes can comprise an effect determinable or measurable using any of a number of metrics, including, for example: a statistically significant reduction (e.g., as compared to the same subject or as compared to a reference subject that did not receive PBM therapy according to the disclosure) in: production of reactive oxygen species (ROS); NFκB transcriptional activity; concentration, activity, and/or expression of ICAM-1; a statistically significant decrease in retinal thickness (e.g., measurable using ocular coherence tomography such as SD-OCT, such as with a Spectralis OCT or TruTrack™ device (Heidelberg Engineering, Heidelberg, Germany)); a reduction (e.g., as compared to the same subject or as compared to a reference subject that did not receive PBM therapy according to the disclosure) in a rate and/or amount of vascular growth and/or new vasculature in an eye of the subject; a statistically significant improvement in a best corrected visual acuity (BCVA) letter score according to an optometry chart (e.g., an ETDRS chart (Precision Vision, USA)) or a Snellen equivalent thereof; a statistically significant improvement in contrast sensitivity (CS) using, for example, the Functional Acuity Contrast Test (FACT), which can be performed using a chart that includes a series of grating patches with functionally different spatial frequencies; a statistically significant improvement in retinal sensitivity (RS) or fixation stability, e.g., as determined by microperimetry; or a statistically significant improvement in responses to one or more questions from the National Eye Institute VFQ-25 Questionnaire (e.g., Questions from Part II: Difficulty with activities, e.g., Q5-Q14), VFQ25 can be found online at, for example, nei.nih.gov/sites/default/files/nei-pdfs/vfq_sa.pdf); a statistically significant increase in a NADPH reductase activity; and/or a statistically significant increase in a mitochondrial membrane potential. It will be understood that a statistically significant response or improvement can be in response to any functional variant of a herein-described test (e.g., a question in a questionnaire can be worded differently than the specific language used in the VFQ-25, but is still within the scope of the present disclosure when it is substantively the same as a reference question in VFQ-25).

Assays for measuring DNA damage caused by reactive oxygen species are described in, for example, Nita and Grzybowski Oxi. Med. Cell. Longev. Vol. 2016, Article ID 3164734 (dx.doi.org/10.1155/2016/3164734). Other ROS assays are known in the art and include in vitro and intracellular ROS assays (e.g., available from Cell Biolabs, Inc.).

Assays for measuring NFκB activity are known in the art (see, e.g., Joseph O. Trask Jr., “Nuclear Factor Kappa B (NF-κB) Translocation Assay Development and Validation for High Content Screening.” 2012 Oct. 1. In: Sittampalam G S, Grossman A, Brimacombe K, et al., editors. Assay Guidance Manual [Internet]. Bethesda (Md.: Eli Lilly & Company and the National Center for Advancing Translational Sciences; 2004-. Available from: ncbi.nlm.nih.gov/books/NBK100914/), and include commercially available assay kits (e.g., Abcam; product nos. ab210613 and ab133112. mRNA expression of NFκB may also be measured (e.g., by real-time PCR).

Other assays for measuring function of ocular cells, such as Müller glial cells, are provided herein.

Anti-ICAM-1 antibodies are commercially available and can be used, for example, in an in situ, ex vivo, or in vivo assay. mRNA expression of ICAM-1 can also be measured. Indirect measures of ICAM-1 activity, such as OCT imaging that captures retinal vascularization and/or inflammation, can also be used.

A “patient” or “subject” includes an animal, such as a human (any gender), dog, cat, monkey, ape, cow, horse, sheep, lamb, pig, mouse, rat, rabbit or guinea pig. The animal can be a mammal, such as a non-primate or a primate (e.g., monkey, ape, and human). In some embodiments, a subject is a human, such as a human infant, child, adolescent, or adult, such as an adult about 20, 25, 30, 35, 40, 45, 50, 55, 60, 65, 70, 75, 80, 85, 90, 95, 100, 105, or 110 years of age, or more. In some embodiments, a subject is a human of 50, 55, 60, 65, 70, 75, 80, 85, 90, 95, 100, 105, or 110 years of age, or more.

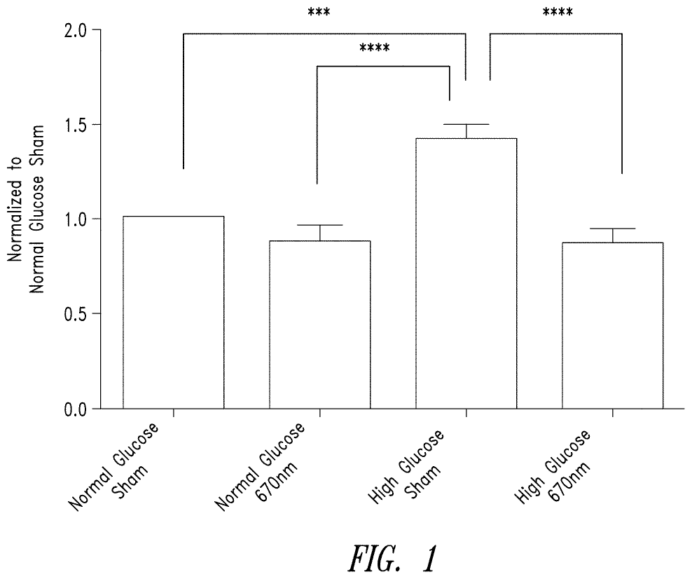

In certain embodiments, prior to administering the PBM light, the subject (e.g., a subject considered at-risk for developing or progressing DR and/or DME): (i) does not present with a mild non-proliferative retinopathy (i.e., small areas of balloon-like swelling in the retina's blood vessels, also referred to as microaneurysms, which may leak fluid into the retina); and/or (ii) does not present with a moderate nonproliferative retinopathy (i.e., swelling and distortion of blood vessels that nourish the retina and/or loss of ability of the blood vessels to transport blood, characterized by changes to the appearance of the retina); and/or (iii) does not present with a severe nonproliferative retinopathy (extensive blockage of blood vessels, depriving blood supply to areas of the retina; secretion of growth factors that signal the retina to grow new blood vessels); and/or (iv) does not present with a proliferative diabetic retinopathy (i.e., proliferation of new blood vessels along an inner surface of the retina and into the vitreous gel; the vessels may leak and bleed; scar tissue may be present and contract; and retinal detachment and vision loss can occur); and/or (v) presents with an early pathological sign of DR and/or DME (e.g., non-central edema, a mild score using a diabetic retinopathy severity scoring system (e.g., a score of below level 43 on using a EDTRS algorithm, (see, e.g., Slakter et al., Trans Am Ophthalmol Soc 113 TD9-1-TD9-18 (2015), in particular the algorithm in FIG. 1; the scoring systems in Slakter et al. are hereby incorporated by reference in their entireties), a ETDRS level of 35, 20, or 10), a EDTRs severity of mild NPDR, very mild NPDR, or no retinopathy, and/or mild quantitative changes in vision such as mile decreases in best corrected visual acuity, contrast sensitivity, microperimetry, electroretinogram or other early diagnostic measures of visual function).

In some embodiments, a subject receiving treatment according to the present disclosure does not develop or progress to, or is delayed in developing or progressing to, mild nonproliferative retinopathy, moderate nonproliferative retinopathy, severe nonproliferative retinopathy, and/or proliferative retinopathy.

In certain embodiments, the subject is, or is suspected of being hyperglycemic (e.g., as compared to a reference glycemic index that is considered normal (“normoglycemic”) for the subject or for a reference subject). Accordingly, in certain embodiments, hyperglycemia is determined by reference to the same subject in a normoglycemic state or by reference to a baseline glycemic state that is considered normal (e.g., for a reference subject); i.e., by a clinician.

In general, a human subject that is hyperglycemic has a blood sugar level higher than 11.1 mmol/l (200 mg/dl), or, in certain embodiments, higher than approximately 15-20 mmol/l (e.g., approximately 250-300 mg/dl). A subject with a consistent blood sugar range between approximately 5.6 and approximately 7 mmol/l (100-126 mg/dl) is considered slightly hyperglycemic, while a subject with a blood sugar level above 7 mmol/l (126 mg/dl) is generally considered to be diabetic.

In certain embodiments, hyperglycemia can alternatively or additionally be indicated by any one or more of: increased thirst and/or hunger; frequent urination; sugar in urine; headache; blurred vision; and fatigue.

In certain embodiments, the subject is, or is suspected of being, diabetic. In certain embodiments, the subject has, or is suspected of having, Type I diabetes. In certain embodiments, the subject has, or is suspected of having, Type II diabetes.

In certain embodiments, the subject had previously received, or is receiving, one or both of an anti-VEGF therapy and a corticosteroid.

In certain embodiments, the eye of the subject had previously experienced onset and/or a progression of DR and/or DME while (e.g., during the course of) or subsequent to receiving a corticosteroid and/or an anti-VEGF therapy, wherein the anti-VEGF therapy optionally comprises an anti-VEGF antibody injection.

In certain embodiments, presently disclosed methods further comprise, following an administration of the PBM light, determining, measuring, or receiving one or more of: (i) a production, expression, and/or activity level in the eye of the subject or in a sample from the eye of the subject, of (a) a reactive oxygen species (ROS); (b) NFκB; (c) ICAM-1; and/or (d) VEGF; (ii) a central retinal thickness of the eye of the subject; or (iii) a best corrected visual acuity (BVCA) score for the subject. In certain further embodiments, if any one of (i)(a)-(iii) is indicative of or associated with the presence or progression of diabetic retinopathy and/or diabetic macular edema, the method comprises administering a further therapy comprising the PBM, an anti-VEGF therapy, a corticosteroid, or any combination thereof.

In some embodiments, prior to administering the PBM, the subject or the eye of the subject (e.g., a cell thereof, such as a Müller glial cell) has increased production, expression, and/or activity of: (i) a reactive oxygen species (ROS); (ii) NFκB; (iii) ICAM-1; (iv) VEGF; or (v) any combination of (i)-(iv), wherein the production, expression, and/or activity is increased as compared to a healthy or reference cell or eye, and wherein the reference cell optionally is or comprises a Müller glial cell.

In certain embodiments, a method further comprises, prior to and/or after administering the PBM light, performing one or more of: an electroretinogram; fluorescein angiography; ocular coherence tomography (OCT) (e.g., spectral domain ocular coherence tomography, enhanced depth imaging OCT, swept source OCT, retinal oximetry, OCT angiography); dark adaptometry; an FST test; or fundus imaging, on the eye of the subject.

### Photobiomodulation (PBM) and PBM Therapy Parameters

“Photobiomodulation,” also referred to as “PBM” herein, refers to the effect of visible light energy, typically of a wavelength from about 500 nm-1000 nm, to stimulate, suppress, or otherwise modulate a biological activity. PBM is distinguishable from other forms of light-based intervention, such as photoablating or photocoagulating lasers, in that it does not cause significant damage to (e.g., cauterize, ablate, coagulate, kill, or scar) to target cells or tissue. Without being bound by theory, PBM may act at the cellular level by activating mitochondrial respiratory chain components, resulting in stabilization of metabolic function. For example, it has been suggested that cytochrome C oxidase (CCO) is a key photoacceptor of light in the far red to near infrared spectral range. Grossman et al., Lasers. Surg. Med. 22:212-218 (1998); Kara et al., J. Photochem. Photobiol. B. 27:219-223 (1995); Karu and Kolyakov, Photomed. Laser Surg. 23:355-361 (2005); Kara et al. , Lasers Surg. Med. 36:307-314 (2005); and Wong-Riley et al., J. Biol. Chem. 280:4761-4771 (2005). Typically, PBM for treating ocular conditions involves delivering light energy from an external light-emitting source to the eye of a subject or patient, wherein the light energy comprises one or more PBM wavelengths and is delivered with sufficient intensity (e.g., power density or irradiance, which may be measured at a target tissue, or an emission source, or at any point therebetween). Standard measurements in this regard include J/cm² and mW/cm². Determination of an appropriate power output to deliver light of appropriate energy(ies) and wavelength(s) to a target of interest can be performed using calculations and methods described in, for example, PCT Publication No. WO 2016/040534A1, the calculations, PBM methods, and devices of which are incorporated herein by reference. In certain embodiments, a target of interest comprises a corneal surface or a retinal tissue in an eye of a subject, such as tissue in and/or around the macula or other retinal tissue. In certain embodiments, a target of interest comprises a Müller glial cell.

Presently disclosed methods comprise use of light having a wavelength in a red wavelength range. In certain embodiments, a wavelength in a red wavelength range is in a range from 620 nm to 750 nm, including any nanometer wavelength therebetween. In further embodiments, a wavelength in the red wavelength range is in a range from 630 nm to 740 nm. In further embodiments, a wavelength in the red wavelength range is in a range from 640 nm to 730 nm. In still further embodiments, a wavelength in the red wavelength range is in a range from 650 nm to 720 nm. In yet further embodiments, a wavelength in the red wavelength range is in a range from 660 nm to 710 nm.

In further embodiments, a wavelength in a red wavelength range is 670±50 nm, 670±40 nm, 670±30 nm, 670±25 nm, 670±20 nm, 670±15 nm, 670±10 nm, or 670±5 nm. In certain embodiments, a wavelength in a red wavelength range is 670 nm.

In some embodiments, the PBM consists essentially of light having a wavelength in a red wavelength range. In some embodiments, the PBM consists of light having a wavelength in a red wavelength range.

In some embodiments, PBM therapy according the present disclosure further includes light of one or more wavelength between 550 nanometers and 1060 nanometers (i.e., 550 nanometers, 1060 nanometers, or a wavelength therebetween). In certain embodiments, PBM therapy according the present disclosure includes light of one or more wavelengths between 550 nanometers and 980 nanometers. In certain embodiments, a method further comprises administering an effective amount of photobiomodulation (PBM) light comprising light having a wavelength in a yellow wavelength range and/or PBM light comprising light having a wavelength in a near infrared (NIR) wavelength range.

In certain embodiments, a wavelength in a yellow wavelength range is in a range from 550 nm to 620 nm. In further embodiments, a wavelength in the yellow wavelength range is in a range from 560 nm to 610 nm. In still further embodiments, a wavelength in the yellow wavelength range is in a range from 570 nm to 600 nm. In particular embodiments, a wavelength in the yellow wavelength range is 590 nm±30 nm, 590 nm±20 nm, 590 nm±15 nm, 590 nm±10 nm, 590 nm±5 nm, or about 590 nm. In certain embodiments, a wavelength in the yellow wavelength range is 590 nm.

In certain embodiments, a wavelength in a NIR wavelength range is in a range from 750 nm to 950 nm. In further embodiments, a wavelength in the NIR wavelength range is in a range from 800 nm to 900 nm. In still further embodiments, a wavelength in the NIR wavelength range is in a range from 825 nm to 875 nm.

In certain embodiments, a wavelength in a NIR wavelength range is 830±50 nm, 830±40 nm, 830±30 nm, 830±25 nm, 830±20 nm, 830±15 nm, 830±10 nm, or 830±5 nm. In certain embodiments, a wavelength in a NIR wavelength range is 830 nm.

In certain embodiments, a wavelength in a NIR wavelength range is 850±50 nm, 850±40 nm, 850±30 nm, 850±25 nm, 850±20 nm, 850±15 nm, 850±10 nm, or 850±5 nm. In certain embodiments, a wavelength in a NIR wavelength range is 850 nm.

In certain embodiments, a method comprises administering to the eye an effective amount of photobiomodulation (PBM) light comprising: (i) light having a wavelength in a red wavelength range; (ii) light having a wavelength in a yellow wavelength range; and (iii) light having a wavelength in a near-infrared (NIR) wavelength range. In further embodiments, a method comprises administering PBM light comprising: (i) light having a wavelength of 590 nm±15 nm; (ii) light having a wavelength of 670 nm±15 nm or 660 nm±15 nm; and (iii) light having a wavelength of 850 nm±15 nm or 830 nm±15 nm.

It will be understood that PBM light of different wavelengths (or in different wavelength ranges) can be administered simultaneously, sequentially, and/or contemporaneously). Accordingly, a method can comprise, for example, administering PBM light comprising light having a wavelength in a red wavelength range, and then administering PBM light comprising light having a wavelength in a yellow range. Alternatively, for example, a method can comprise simultaneous administration of PBM light comprising light having a wavelength in a red wavelength range, and PBM light comprising light having a wavelength in a yellow range.

Other parameters of PBM therapy according to the present disclosure include, for example: light emission; power density; pulsing or continuous light delivery; length of pulsed light; width of a pulsed light beam; temporal pulse shape(s), duty cycle(s), pulse frequency(ies); irradiance per pulse; beam diameter; sequence and number of exposures to the or more administered PBM lights or wavelengths; duration of a exposure to the one or more administered PBM lights or wavelengths; duration of a treatment session; whether a subject's eye is open or closed during all or part of a treatment; or the like.

For example, in certain embodiments, any PBM light or wavelength of the present disclosure may be emitted from a source at an intensity (e.g., power density) of from about 0.001 mW/cm² to about 100 mW/cm² or more; e.g., about 0.001, 0.005, 0.01, 0.05, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200 or more mW/cm², or any integer or non-integer value therewithin.

In some embodiments, any PBM light or wavelength of the present disclosure may have a fluence and/or an intensity at an emission surface of a light source in a range from about 0.1 nJ/cm² to about 50 J/cm², in a range from about 0.1 mJ/cm² to about 20 J/cm², in a range from about 0.1 mJ/cm² to about 10 J/cm², in a range from about 1 J/cm² to about 20 J/cm², in a range from about 1 J/cm2 to about 10 J/cm², or about 1, 1.5, 2, 2.5, 3, 3.5, 4, 4.5, 5, 5.5, 6, 6.5, 7, 7.5, 8, 8.5, 9, 9.5, 10, or more J/cm².

In embodiments comprising administration of PBM light of multiple wavelengths, two or more of the lights can be administered (or emitted from a light source) at the same or at different intensities.

In certain embodiments, for example, a method of the present disclosure comprises light comprising a wavelength in a red wavelength range, wherein the light comprising the wavelength in the red wavelength range is emitted from a source at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more mW/cm². It will be appreciated that in embodiments further comprising administration of light having a wavelength in a yellow wavelength range, a NIR wavelength range, or both, either or both of the yellow-wavelength or the NIR-wavelength can also be emitted from a source at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more mW/cm².

It will be understood that in any of the presently disclosed methods, a light that comprises a PBM wavelength in a particular range (e.g., red, yellow, or near infrared) or comprises a particular PBM wavelength or range (e.g., 670 nm, 590 nm, 830 nm, 850 nm) can be partially, substantially, or entirely composed of light of the PBM wavelength or wavelength range. In other words, a recited PBM wavelength or wavelength range can account for, e.g., about 5%, 10%, 20%, 30%, 40% , 50% , 60% , 70%, 80%, 90%, 91%, 92%, 93%, 94%, 95%, 96%, 97%, 98%, 99%, or more of the light. In any embodiment comprising use of multiple (i.e., two or more) wavelengths, any two or more of the multiple wavelengths can be emitted or delivered simultaneously, concurrently, or in any sequence, including overlapping and non-overlapping sequences and sequences wherein a resting period comprising no PBM light, or PBM light of a different wavelength, is interspersed between the emission or delivery of the two or more wavelengths.

In any of the embodiments disclosed herein, a PBM light can be delivered in a pulsed fashion, a continuous fashion, or both. Pulsed light can be delivered in any shape, frequency, irradiance, duty cycle, or other parameter appropriate to a treatment. In some embodiments, PBM light is pulsed at a frequency of about 1 Hz to 100 Hz, from about 100 Hz to about 1 kHz, less than 1 Hz, or more than 100 Hz. In embodiments that comprise two or more PBM wavelengths, any two or three wavelengths may be delivered to a subject or subject eye in a pulsed or a continuous fashion. In some embodiments, one of: a wavelength in the red wavelength range, a wavelength in the yellow wavelength range, and a wavelength in the NIR wavelength range are delivered at least in part in a pulsed fashion; and another of a wavelength in the red wavelength range, a wavelength in the yellow wavelength range, and a wavelength in the NIR wavelength range are delivered at least in part in a continuous fashion.

In certain embodiments, a PBM light according to the present disclosure has any beam diameter that is suitable to reach and sufficiently contact a target area (e.g., cell, organ, body party, or tissue). A beam diameter can be measured at a treatment pane, at the point of exit or emission from a light source, or at any point therebetween. Unless otherwise indicated, a diameter of a light beam as described herein refers to the diameter at a treatment plane. Suitable beam diameters and light intensities can be readily determined by a person of ordinary skill in the art in regard to, for example, the particular cell, organ, body part, or tissue to be targeted, the type, severity, and stage of disease or condition, the size, age, eye (iris) color of the subject, the distance from the point of light emission to the target cell, organ, body part, or tissue, or the like. For example, in certain embodiments, a beam for providing PBM light to a retinal tissue in an eye of a subject having or suspected of having dry age-related macular degeneration can be about 10, 15, 20, 30, 35, 40, 45, or more mm in diameter. In particular embodiments, a beam has a diameter of about 30 mm.

Treatment exposure times will also be readily determined by those of ordinary skill in the art with regard to, for example, the particular relevant feature(s) of the subject, the disease or condition to be treated, the type, intensity, and/or wavelength(s) of light being administered, or the like. In some embodiments, a treatment can comprise administering one or more light comprising a PBM wavelength for between about 0.0001 milliseconds and about 1 hour, or more. In certain embodiments, a treatment session comprises administering one or more PBM light, wherein each of the one or more light is administered for about 1, 5, 10, 15, 20, 25, 30, 35, 40, 45, 50, 55, 60, 65, 70, 75, 80, 85, 90, 95, 100, 105, 110, 115, 120, 130, 140, 150, 160, 170, or 180 seconds or more. In some embodiments, a total time of a treatment session comprises the combined duration of the light administration(s), and can range from less than about one minute to ten or more minutes. In particular embodiments, a treatment session comprises a total treatment (e.g., exposure) time of less than about 20 minutes, less than about 15 minutes, less than about 10 minutes, or less than about 9, 8, 7, 6, 5, 4, 3, or 2 minutes, or less than about one minute. In some embodiments, a treatment session comprises a total treatment (e.g., exposure) time of less than about 5 minutes, or is about 4 minutes. The total treatment time can refer to treatment of a single eye (even if more than one eye of a subject is to receive treatment in the session) or of more than one eye.

In certain embodiments, a method comprises administering PBM light on 1, 2, 3, 4, 5, 6, or 7 days in a one-week (7-day) period. In certain embodiments, a method comprises administering PBM light one or more times per week for 2 or more (e.g., 2, 3, 4, 5, 6, 7, 8, 9, 10, 11, 12, 13, 14, 15, 16, 17, 18, 19, or more consecutive weeks). In certain embodiments, a method comprises administering PBM light monthly, bimonthly, or once every three, four, five, six, seven, eight, nine, ten, eleven, or twelve months.

In any of the herein described embodiments, a method can comprise a treatment session that includes one or more phases, wherein the one or more phases are each characterized by any one or a combination of the herein described PBM parameters; e.g., light color, light wavelength, pulsed versus continuous light, light intensity, light delivered to an open eye, light delivered to a closed eye through an eyelid, light delivered to a first eye and optionally to a second eye, a resting period wherein no light or no PBM light is being administered or delivered, varying intensities of the light(s), or the like.

For example, in certain embodiments, a method can comprise a first phase comprising administering light (e.g., one or two or more beams) comprising a first PBM wavelength, and a second phase comprising administering light comprising a second, or a second and a third, PBM wavelength. It will be understood that phases can comprise lights, wavelengths, continuous versus pulsing, rest times, changes in intensity, or the like in any sequence and/or combination. In general, the lights, wavelengths, light intensities, treatment times, phases, beam diameters, pulsing and/or continuous delivery during a treatment session according to the presently disclosed methods will be selected according to their efficacy or potential efficacy for treating the given subject and/or condition, by the convenience and comfort threshold for the subject (e.g., not keeping an eye open for more than few minutes to receive PBM), and by safety considerations (e.g., using light of an appropriate intensity for PBM, and avoiding undesirable heating, ablation, cauterization, coagulation, or other forms of damage). Non-limiting examples include all of the PBM treatment parameters, and the specific treatment methods and combinations of the PBM treatment parameters described in PCT Patent Publication No. WO 2016/040534A1, which are incorporated herein by reference.

In specific embodiments, a method comprises administering PBM light for a about 180 or more seconds, wherein the method comprises one or more phases that each independently comprise a continuous administration of PBM, pulsed PBM, or both, and can include a wavelength in one, two, or three of a red wavelength range, a yellow wavelength range, and a near infrared wavelength range.

### Devices

Light suitable for PBM can be produced, for example, by a laser (e.g., a low-power laser) or a non-coherent light source (e.g., a light emitting diode (LED), a laser diode (e.g., a gallium-aluminum-arsenic (GaAlAs) laser diode, an aluminum gallium indium phosphide (AlGaLnP) laser diode, a diode-pumped solid state (DPSS) laser, a vertical cavity surface-emitting laser (VC SEL) diode, or the like), a lamp, or the like).

Any suitable light-emitting device can be used to provide PBM therapy of the present disclosure. It will be understood that PBM light can be generated and/or administered using a single device or source or using different devices or sources during a treatment session or over the course of a treatment regimen (e.g., comprising multiple treatment sessions). Exemplary devices include all of those disclosed in PCT Publication No. WO 2016/040534A1, as well as those comprising combinations of the features disclosed therein, (see also the VALEDA™ Light Delivery System by LumiThera), and in US Pat. No. 9,592,404. Other devices include the Warp 10™ (Quantum Devices, Inc.; Barneveld, Wis.) and the GentleWaves® (Light Bioscience LLC; Virginia Beach, Va.) instruments. Accordingly, contemplated herein are devices including self-standing devices, wearable devices (e.g., in the form of glasses-like devices, which may be binocular or monocular, eye-patch-type devices, masks, or the like), hand-held devices, and the like.

In certain embodiments, a device comprises a microprocessor or microcontroller that modulates one or more parameter of PBM therapy; e.g., any one or more of the parameters described herein. In some embodiments, a device is programmable and can, for example, provide a PBM therapy that is customized or tailored for a particular subject, subject eye, or group, class, or category of subjects. Therapeutic settings (e.g., parameters) for providing PBM to a subject can be adjusted during a treatment session (e.g., in real time), or between treatment sessions, or over the course of a treatment regimen, regime, or program based on. Exemplary microprocessors and devices, systems, and methods comprising use of the same for providing PBM therapy are described in PCT Publication No. WO 2016/040534A1, and are incorporated herein.

Also provided are devices and systems useful for imaging, gathering or capturing data from, and/or processing information about an eye of a subject. Suitable devices include, for example, fundus cameras and related filters, ocular coherence tomography devices, ERG machines, dark field adaptometers (e.g., Labrique et al., BMC Opthnalmol. 15:74 (2015); AdaptDx®), and multimodal imaging devices (e.g., Spectralis® and related modules).

### EXAMPLE

### In Vitro Model of PBM Therapy for Diabetic Retinopathy

Müller glial cells have been shown to play a primary role in the progression of DR due to a shift in their physiology from an anti-inflammatory to a pro-inflammatory state. Experiments assessing the ability of PBM (670 nm) to improve cellular function under high glucose conditions were performed in cultured retinal Müller glial cells.

Methods

Cell Line: Experiments were performed on rat Müller glial cells obtained from Dr. John Mieyal, Case Western Reserve University. Müller cells were exposed to high glucose (25 mM) or normal glucose (5 mM) Dulbecco's modified Eagle's medium (Invitrogen cat. nos. 11995 and 11885, respectively) to mimic hyperglycemic and normal conditions. Cell medium was changed daily to maintain constant glucose load. For mitochondrial assays and oxidative stress assays, Müller cells were grown in a phenol free DMEM normal (5 mM) or high glucose media (25 mM). Phenol free normal glucose media (Invitrogen cat. no. 11054-020) was supplemented with 5% L-glutamine and high glucose phenol free media (Invitrogen cat. no. 21063-029) was supplemented with 110 mg/L sodium pyruvate to sustain Müller cell growth in culture.

Light Treatment: Cell cultures were exposed to 670 nm light from a light emitting diode (LED) array (10 cm×25 cm) (Quantum Devices Inc. Barneveld, Wis.) positioned on top of the culture plate at a dose of 25 mW/cm² for 180 seconds (total of 4.5 J/cm²). Sham-treated cells were handled in a similar manner except that the LED array was not illuminated. PBM-treatment occurred 1 time per day over 24 hours or 72 hours, depending on the assay. All assays were conducted 1 hour following the final light treatment.

MTT Assay: Cells were plated in triplicate in 96-well plates at a density of 30,000 cells/well, cultured in the respective high or normal glucose media conditions. At 24 hours, respective media were exchanged and cells were incubated for 1 hour. After 1 hour, cells were treated with either 670 nm light or sham light for 180 seconds. After 1 hour, media was removed, and cells were washed with 1× PBS. Then, cells were incubated with 5 mg/mL MTT in medium for 3 hours at 37° C. MTT solution was solubilized in 10% Triton x100 and isopropanol with 0.1N HCl (final concentration) and was shaken for 15 mins in the dark. Each well was thoroughly mixed and absorbance was read at 590 nm (Biotek Synergy™ HT).

TMRE Mitochondrial Membrane Potential: Cells were plated in triplicate in 96-well plates at a density of 30,000 cells/well in normal or high glucose conditions for 24 hours. At 24 hours, respective media were exchanged and incubated for 1 hour. After 1 hour, cells were treated with either 670 nm light or sham light for 180 seconds. After 1 hour, media was removed, and cells were washed with 1× PBS. The TMRE assay (Abcam, Cambridge, Mass.) proceeded per manufacturer's instructions. Briefly, cells were incubated in 200 nM TMRE dissolved in their respective cell mediums for 30 mins. Cells were washed with PBS and fluorescence (EX/EM: 549/575 nm) was read in 100 μL PBS/0.2% BSA solution.

ATP Measurement: Cells were plated in triplicate in 96-well opaque-walled plates at a density of 30,000 cells/well in phenol free normal or high glucose conditions for 24 hours. At 24 hours, respective media were exchanged and cells were incubated for 1 hour. The cells were treated with 670 nm light (4.5 J/cm²) or sham light for 180 seconds. After 30 mins., plates were removed from 37° C. and incubated at room temperature for another 30 mins. Then, an equal volume of CellTiter-Glo® 2.0 Reagent (100 μL) (Promega®) was added to Müller cells in their respective media (100 μL). Plates were shaken for 10 minutes at room temperature. Luminescence was read (gain 115-135) and compared to an ATP standard curve.

Assessment of ROS: Cells were plated in triplicate in 96-well plates at a density of 30,000 cells/well in normal or high glucose conditions for 24 hours. At 24 hours, respective media were exchanged and cells were incubated for one hour. The cells were treated with 670 nm light (4.5 J/cm²) or sham light for 180 seconds. One hour after light treatment, cells were washed with 1× PBS and a 40 mM DCFDA solution (in respective media) was overlaid immediately, per manufacturer's instructions (Cayman Chemical Inc., Ann Arbor, Mich.). DCFDA solution was allowed to incubate at 37° C. for 1 hour. After 1 hour, DCFDA solution was removed and replaced with 100 μL of a 1× buffer and read via fluorescence in a plate reader with EX/EM 484 nm/528 nm with a gain of 100-120.

Measurement of NFκB activity: NFκB activity was measured from analysis of 20,000 Müller cells/well (6-well dish) grown for 3 days. At day 3, cells were approximately at a confluency of 60-70%. Cells were washed twice with 1× PBS. Cells were then co-transfected according to manufacturer instructions with a cocktail containing 1 ml/well Opti-MEM, 5 μl/well lipofectamine 2000, 1 μg/well NFκB plasmid and 0.1 μg/well Renilla plasmid (Shelton et al. 2007). Transfection was allowed to proceed for 8 hours. Post-transfection, the transfection cocktail was aspirated and replaced with DMEM, 5 mM glucose, 2% FBS, 1% penicillin streptomycin, 110 mg/L sodium pyruvate for 3-4 hours. Cells were then switched to appropriate treatment media (high (25 mM) or normal (5 mM) glucose). Cells were maintained in either high or normal glucose conditions for 3 days and were either treated with 670 nm light (4.5 J/cm²) or sham treated for 180 sec daily. At completion of the treatment, lysates were collected in 1× passive lysis buffer (PLB) and assessed via a Dual Luciferase Reporter assay (Promega® cat. no. E1910). Luminescence measured was normalized to Renilla.

Assessment of ICAM-1: Cells were cultured and treated with light 1×/day for 5 days. One hour after treatment with light on day 5, cells were harvested and lysed on ice for 15 min in 1× RIPA buffer (Alfa Aesar, cat. no. J60629) containing 1× protease inhibitor cocktail (Thermo Scientific, cat. no. 87786). 100 μg of cell lysate was boiled at 95° C. for 15 min in Laemilli buffer (10% glycerol, 2% SDS, 0.01% bromophenol blue, 200 mM Tris HCl pH 6.8, 20 mM DTT). Samples were run on a SDS-PAGE gel and transferred to a polyvinyl difluoride (PVDF) membrane. Prior to transfer, the membrane was soaked in methanol for 2 minutes, then washed with Transfer Buffer (0.3% w/v Tris, 1.45% w/v glycine, and 10% v/v methanol). Anti-rabbit ICAM-1 (Cell Signaling, cat. no. 49155) and anti-rabbit GAPDH (Cell Signaling, cat. no. 21185) were used to probe the membrane at 1:500 and 1:5000, respectively. An anti-goat, anti-rabbit horseradish peroxidase-linked secondary antibody (1:10,000, Jackson Labs, catalog number 111-035-144) was added, followed by detection using SuperSignal™ Chemiliuminescent HRP Substrate and exposure to film. ImageJ software was used to compare protein levels normalized to GAPDH.

VEGF qPCR: Müller cells were grown in culture for 3 days in a 10 cm dish. Each plate was treated with either sham or 670 nm light at an intensity of 4.5 J/cm². Cells were washed with 1× PBS and harvested in Trizol. RNA was extracted per manufacturer's instructions. Biorad iScript cDNA kit was used to create cDNA. qPCR was performed using Biorad SybrGreen Super Mix on a step1plus machine following manufacturer's instructions.

Primers used were for VEGF
(FP CCGCAGACGTGTAAATGTTCC (SEQ ID NO.: 1),
RP ACGGTGACGATGGTGGTGT (SEQ ID NO.: 2))
and actin
(FP: TGACGTGGACATCCGCAAAG (SEQ ID NO.: 3),
RP: CTGGAAGGTGGACAGCGAGG (SEQ ID NO.: 4)).

Samples were run in triplicate and normalized to the sham, normal glucose control. Values were calculated via ddCt.

Statistical Analysis: Samples were measured in duplicate or triplicate. All glucose and light conditions were normalized to the normal glucose sham condition. Averages, standard deviations and standard errors were calculated. Differences between each sample group were analyzed by ANOVA, followed by Bonferroni post-hoc testing. The alpha for statistical analysis was set at a p value of 0.05.

Results

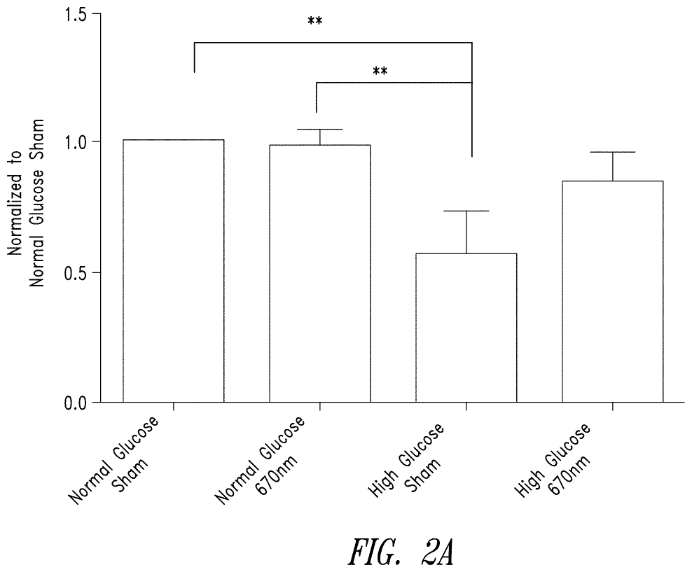

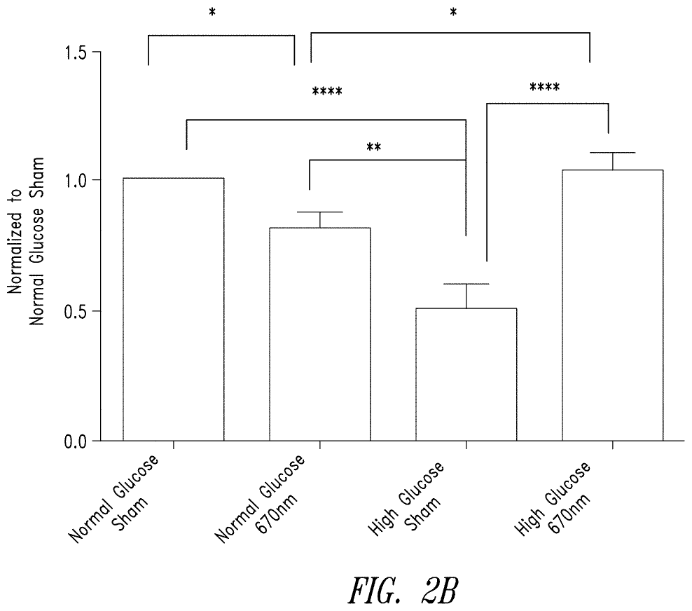

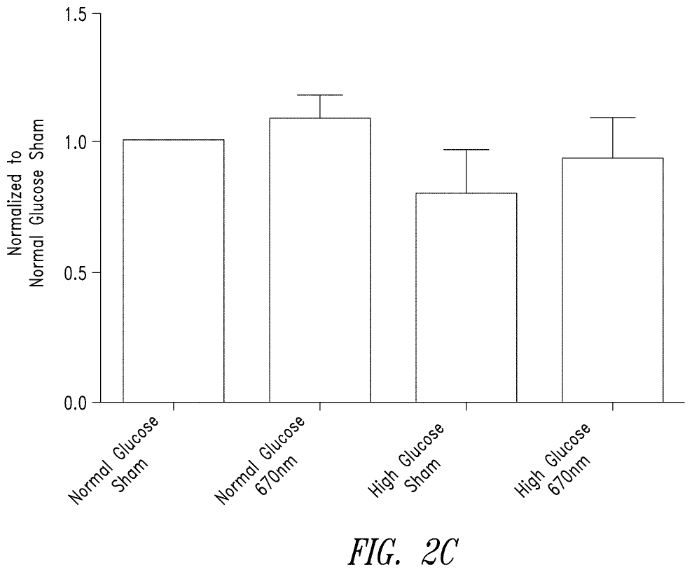

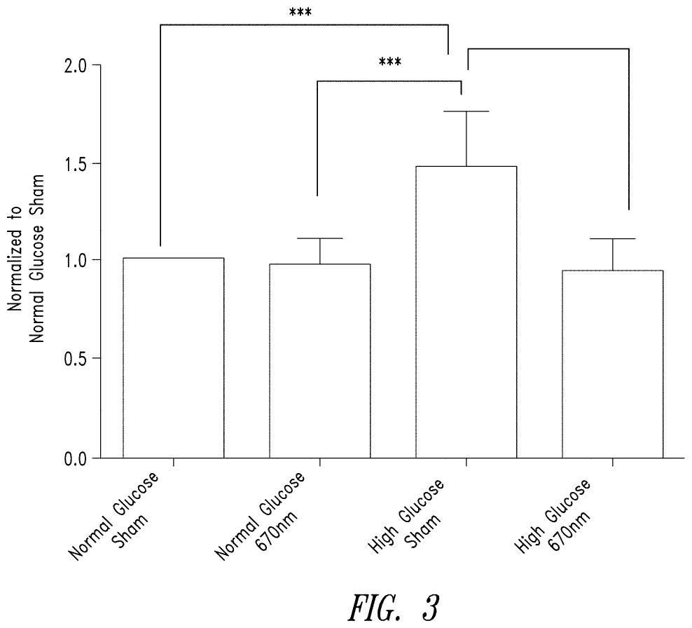

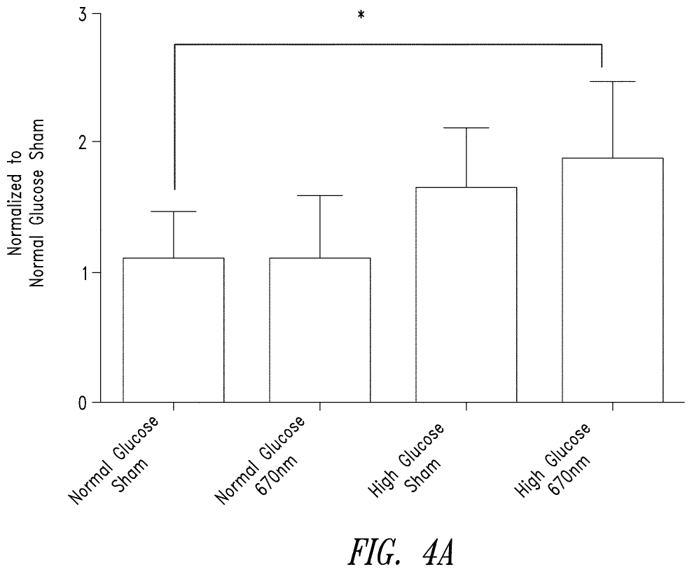

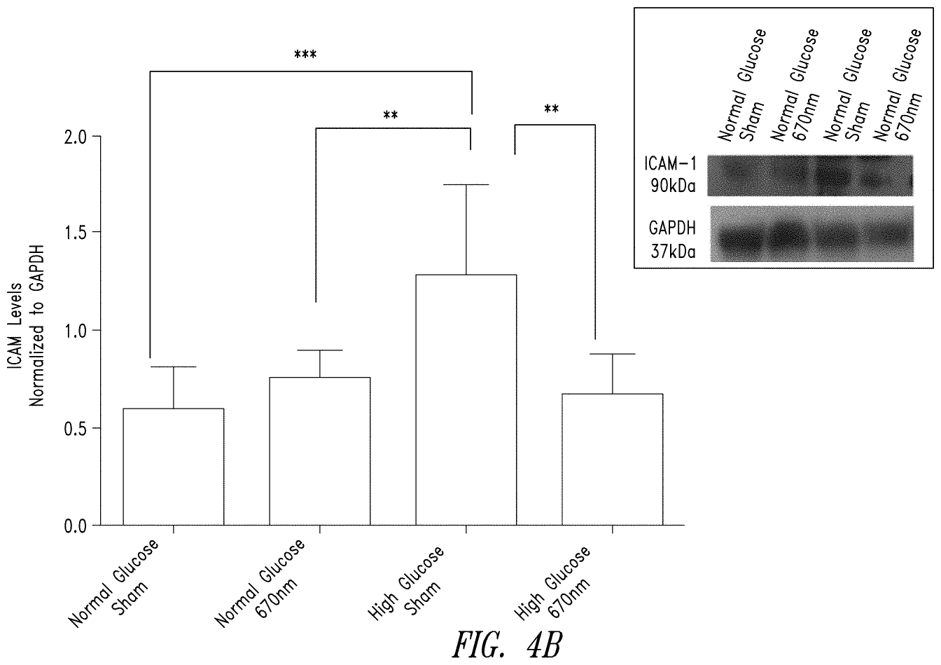

Retinal Müller glial cells are among the first cells to exhibit metabolic changes in response to retinal stress and disease and play a key role in the pathophysiology of diabetic retinopathy. These data show that exposure to high glucose conditions increased ROS production and disrupted mitochondrial bioenergetics, followed by activation of the NFκB signaling pathway, culminating in an increase in intracellular adhesion molecule 1 (ICAM-1) and VEGF. Treatment with 670 nm PBM (4.5 J/cm²) reduced ROS concentrations (FIG. 1), restored mitochondrial bioenergetics (FIGS. 2A-2C), blocked activation of the NFκB signaling pathway (FIG. 3), and prevented increases in ICAM expression (FIG. 4B). These studies demonstrate that 670 nm PBM attenuates glucose-induced disruptions in cellular metabolic activity, beginning with ROS generation and mitochondrial redox changes, followed by modified intracellular signaling and culminating in increased production of angiogenic and inflammatory mediators characteristic of diabetic retinopathy. Importantly, these findings support using PBM early in the pathogenesis of diabetic retinopathy, in contrast to other therapeutic approaches which act much later in the course of disease.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for preventing or delaying onset and/or progression of (i) diabetic retinopathy and/or (ii) diabetic macular edema in an eye of a subject, the method comprising administering to the eye an effective amount of photobiomodulation (PBM) light comprising light having a wavelength in a red wavelength range.

2. The method of claim 1, wherein, prior to administering the PBM light, the subject:
(i) does not present with a mild non-proliferative retinopathy; and/or
(ii) does not present with a moderate nonproliferative retinopathy; and/or
(iii) does not present with a severe nonproliferative retinopathy; and/or
(iv) does not present with a proliferative diabetic retinopathy; and/or
(v) presents with an early pathological sign of DR and/or DME.

3. The method of claim 1, wherein the red wavelength range is 670±50 nm.

4. The method of claim 1, wherein the red wavelength range is 670 nm±30 nm.

5. The method of claim 1, wherein the PBM consists essentially of light having a wavelength in a red wavelength range.

6. The method of claim 1, wherein the PBM consists of light having a wavelength in a red wavelength range.

7. The method of claim 1, further comprising administering to the eye of the subject an effective amount of PBM comprising:
(i) light having a wavelength in a yellow wavelength range; and/or
(ii) light having a wavelength in a near-infrared (NIR) wavelength range.

8. The method of claim 1, wherein the subject is hyperglycemic.

9. The method of claim 1, wherein the subject has, or is suspected of having, Type I diabetes.

10. The method of claim 1, wherein the subject has, or is suspected of having, Type II diabetes.

11. The method of claim 1, wherein the subject had previously received, or is receiving, one or both of an anti-VEGF therapy and a corticosteroid.

12. The method of claim 1, wherein the eye of the subject had previously experienced onset and/or progression of DR and/or DME while or subsequent to receiving a corticosteroid and/or an anti-VEGF therapy.

13. The method of claim 1, wherein the method comprises:
(i) administering the PBM light to the eye at a dose of at least about 4.5 J/cm²;
(ii) administering the PBM light in a dose comprising about 25 mW/cm² for about 180 seconds;
(iii) administering the PBM light through a closed eyelid of the subject; or
(iv) any combination of (i)-(iii).

14. The method of claim 1, wherein the method comprises administering the PBM light on one or more days in a one-week period.

15. The method of claim 14, comprising administering the PBM light one or more times per week for two or more consecutive weeks.

16. The method of claim 1, further comprising, following an administration of the PBM light, measuring one or more of:
(i) a production, expression, and/or activity level in the eye of the subject or in a sample from the eye of the subject, of
(a) a reactive oxygen species (ROS);
(b) NFκB;
(c) ICAM-1; and/or
(d) VEGF;
(ii) a central retinal thickness of the eye of the subject; or
(iii) a best corrected visual acuity (BVCA) score for the subject.

17. The method of claim 16, wherein if any one of (i)(a)-(iii) is indicative of or associated with the presence or progression of diabetic retinopathy and/or diabetic macular edema, the method comprises administering a further therapy comprising the PBM.

18. The method of claim 1, further comprising, prior to and/or after administering the PBM light, performing one or more of: an electroretinogram; fluoroscein angiography;
ocular coherence tomography (OCT) (e.g., spectral domain ocular coherence tomography, enhanced depth imaging OCT, swept source OCT, retinal oximetry, OCT angiography); dark adaptometry; an FST test; or fundus imaging, on the eye of the subject.

19. A method for treating (i) diabetic retinopathy (DR) and/or (ii) diabetic macular edema (DME) in an eye of a subject, the method comprising administering to the eye an effective amount of photobiomodulation (PBM) light comprising light having a wavelength in a red wavelength range.

20. A method for:
(i) reducing or modulating, in an eye of a subject: (a) oxidative stress; (b) NFκB activity; and/or (c) ICAM-1 expression or activity; and/or
(ii) increasing or modulating, in an eye of a subject, a mitochondrial membrane potential, a NADPH-dependent oxidoreductase activity level, or both,
the method comprising administering to the eye an effective amount of photobiomodulation (PBM) light comprising light having a wavelength in a red wavelength range.
